(19) United States
(12) Patent Application Publication
(10) Pub. No.: US 2023/0320941 A1
(43) Pub. Date: Oct. 12, 2023

(54) DENTAL COMPOSITION, KIT AND METHOD FOR USE AS ALTERNATIVE RESTORATIVE MATERIAL TO DENTAL AMALGAM

(71) Applicant: SDI Limited, Bayswater (AU)

(72) Inventor: Paul FARRAR, Bayswater (AU)

(21) Appl. No.: 18/021,149

(22) PCT Filed: Aug. 13, 2021
(86) PCT No.: PCT/AU2021/050894

§ 371 Date: Feb. 13, 2023

(30) Foreign Application Priority Data
Aug. 13, 2020 (AU) 2020902880

(51) Int. Cl. A61K 6/887; A61K 6/61; A61K 6/831; A61K 6/84

(52) U.S. Cl. CPC A61K 6/887; A61K 6/61; A61K 6/831; A61K 6/84

(57) ABSTRACT

The present invention relates to a dental composition and a dental kit comprising a polymerisable adhesive free of a photoinitiator, and a self-cure composite restorative including a redox initiator system. The dental composition cures upon contact between the self-cure composite restorative and the adhesive, to thereby form a dental restoration. The present invention also relates to a method of applying the dental composition, and methods of replacing a dental amalgam with the dental composition, or dental kit, of the present invention.

### FIELD OF THE INVENTION

The present invention relates to a dental composition, kit and method for use in restoration of tooth cavities. More particularly, the present invention relates to a dental composition as an alternative restorative material to dental amalgam.

### BACKGROUND

In the field of dentistry, cavities are openings or holes in teeth brought on by tooth decay. Restoration of teeth with dental cavities typically involves filling the cavity with a dental amalgam in order to repair the tooth structure.

Dental amalgam is a reliable and long-lasting material for restoring large dental cavities caused by tooth decay. Amalgam is often the material of choice due to its low cost, high strength, ease of placement and proven clinical longevity. Unfortunately, amalgam has several disadvantages that are leading to a decline in its use. Firstly, dental amalgam does not adhere to the tooth surface so large undercuts are required to ensure retention of the restoration. This method of retention means that excessive healthy tooth structure is undesirably removed. Secondly, the amalgam fillings tend to expand and contract with heat and cold, which can fracture the tooth containing the filling. Thirdly, amalgam's silvery/grey appearance does not match that of dentin and enamel, leading to a poor aesthetic match with the surrounding tooth, subsequently resulting in poor patient satisfaction.

Moreover, a conventional amalgam composition can contain up to 50 percent mercury, a highly toxic heavy metal. Although when the amalgam filling is set, there is no liquid mercury present as the mercury is bound in an intermetallic compound co-formed with silver, tin and copper, research has shown that mercury can still leak in a vapour form from amalgam. This filling material also tends to corrode overtime, causing mercury to be released from the filling material. In addition to health concerns associated with amalgam, various international regulatory bodies are also in the process of phasing down or phasing out dental amalgams in their respective regions due to global concerns of mercury in the environment.

To date, dental restorative compositions or products which do not involve amalgam have been developed. These compositions or products generally involve light polymerisable dental materials. For example, JP2010280630A discloses a two-part light-curable dental adhesive product that is mixed prior to application on the tooth. U.S. Pat. No. 4,820,744 discloses a similar two-part dental restorative material. JP2019112343 discloses a one-part dental composition which can either be an adhesive or bonding agent which includes photopolymerisation initiators in the formulation.

With the light-cured restorative compositions or products, there are concerns about the depth of appropriate curing, which is a crucial factor affecting the physical and mechanical properties of the restorative material(s) involved. Inadequate polymerisation may result in greater deterioration of a restorative material, decreased bond strength between the tooth and the restorative material, and reduced hardness/endurance of the dental restorative.

The light-cured restorative compositions or products typically require a light-curing unit in order to deliver sufficient energy to enable curing to take place and to control the depth of curing. The light-curing or photopolymerisation process will depend on both the light intensity and light-curing time. These factors can be affected by many variables, such as the operator (e.g. a dentist), the manner of using the light-curing unit (e.g. relative distance to the material to be cured), the condition of the light-curing unit (e.g. battery, cover and/or curing-tip conditions) and power supply quality to the light-curing unit. Any single one, or a combination, of these factors can lead to an inadequate level of conversion of a monomer to a polymer and/or inadequate depth of curing, resulting in restorative materials with poor physical and/or mechanical properties.

The existing light polymerisable dental compositions may also require the preparation of a mix containing two or more parts to first form an adhesive component or a bonding agent. This external mixing step can be messy or fiddly, particularly when a part is quick to set.

Existing dental compositions or products also tend to involve amine-based polymerisation initiators which can cause discolouration of the final restoration.

The present invention attempts to provide a restorative solution which overcomes at least in part the aforementioned problems or disadvantages associated with existing dental restorative compositions or materials.

### SUMMARY OF INVENTION

In accordance with a first aspect of the present invention there is provided a dental composition comprising
a polymerisable adhesive free of a photoinitiator, and
a self-cure composite restorative including a redox initiator system;
wherein the dental composition cures upon contact between the self-cure composite restorative and the adhesive, to thereby form a dental restoration.

In accordance with a second aspect of the present invention, there is provided a dental kit with pre-packaged parts for restoring a tooth cavity, the kit comprising:
a polymerisable adhesive free of a photoinitiator; and
a self-cure composite restorative including a redox initiator system.

In a preferred embodiment, the adhesive in the first or second aspect of the present invention is a one-part adhesive, comprising at least one reducing agent. Preferably, the adhesive comprises at least one polymerisable component and at least one reducing agent.

In a preferred embodiment, the self-cure composite restorative in the first or second aspect of the present invention is a two-part restorative, or two-part combined (or mixed) restorative, with one part including at least one oxidising agent of the redox initiator system, and one part including at least one reducing agent of the redox initiator system. In a preferred embodiment, the redox initiator system in the first or second aspect of the present invention is an amine-free, or substantially amine free, redox initiator system. In a preferred embodiment, the composite restorative comprise(s) at least one polymerisable component.

In accordance with a third aspect of the present invention there is provided a method of applying a dental composition to a tooth cavity, the method comprising the steps of:
applying a polymerisable adhesive free of a photoinitiator to the cavity to form an adhesive layer; and
applying a self-cure composite restorative including a redox initiator system over the adhesive layer.

In a preferred embodiment, the self-cure composite restorative comprises at least one oxidising agent and at least one reducing agent for the redox initiator system.

In a preferred embodiment, the polymerisable adhesive and self-cure composite restorative in the third aspect of the present invention are those according to the first or second aspect of the present invention.

In accordance with a fourth aspect of the present invention, there is provided a method of restoring a tooth structure using a dental composition or a dental kit according to the first or second aspect of the present invention.

In accordance with a fifth aspect of the present invention, there is provided a method of replacing a dental amalgam material for tooth restoration by the use of a dental composition according to the first aspect of the present invention, or a dental kit according to the second aspect of the present invention, which is able to form or provide a dental restoration in situ free of the amalgam material.

In accordance with a sixth aspect of the present invention, there is provided a method of replacing a dental amalgam filling within a tooth cavity by: a) removing the dental amalgam filling from the tooth cavity, and b) forming a dental composition according to the first aspect of the present invention within the same tooth cavity.

In accordance with a seventh aspect of the present invention, there is provided a use of a dental kit according to the second aspect of the present invention in the preparation of a dental composition for restoring a tooth structure.

### DESCRIPTION OF EMBODIMENTS

### Definitions

The terms “comprising”, “including”, “having”, or variations thereof, as used herein are open-ended terms meaning ‘containing but not limited to’ the feature(s) recited after the term. Whilst not limiting, these terms also encompass the more restrictive meanings of “consisting essentially of” and “consisting of”.

The term “composition” as used herein refers to a mixture, composite or system of individual components/constituents/substances/ingredients, which are, or may be, combined to form a whole, or form a dental composition in accordance with the present invention. In a preferred embodiment, a dental composition of the present invention may be formed, in situ, from components/constituents including an adhesive and a self-cure composite restorative of the present invention.

The term “adhesive” as used herein refers to a component of the dental composition or kit of the present invention that is able to adhere to a tooth structure, cavity or cavity interface, and to the self-cure composite restorative of the present invention. The term adhesive may be used interchangeably with terms such as “primer” or “bonding agent”.

The term “composite restorative” as used herein means a restorative in the field of dentistry for restoring the form or function of a damaged tooth or damaged teeth, the restorative being composed of two or more parts, components or materials. For example, the composite restorative may comprise various organic and inorganic ingredients/materials, including one or more polymerisation initiators and one or more filler materials. In a preferred embodiment of the present invention, the composite restorative has two parts, each part itself may comprise various organic and inorganic ingredients/materials, including each part comprising at least one polymerisation initiator to constitute the redox initiator system of the present invention. The composite restorative may take various forms including liquid, powder, resin/paste, or a combination of these forms (e.g. with one part in a liquid form, another in a powder form, or both in a resin/paste form). In a preferred embodiment with the two-part composite restorative, the two parts may be separately contained in two different containers, or contained in the same container (e.g. a capsule, capsule tray or filled syringe) that is compartmentalised.

The term “self-cure” as used herein means that the restorative/composite restorative of the present invention is able to chemically polymerise and/or cure without relying on a photoinitiator and/or photoirradiation. In particular, the self-cure composite restorative of the present invention relies on a redox cure system to achieve (sufficient) polymerisation/curing.

The term “redox initiator system” as used herein refers to a free-radical initiator system in which reactive species which permit the polymerisation of a polymerisable component in the composite restorative and/or the adhesive of the present invention are generated through one or more redox reactions requiring at least one oxidising agent and at least one reducing agent. In one embodiment of the present invention, whilst the composite restorative includes both the oxidising and reducing components of the redox initiator system, the adhesive also comprises one of the oxidising or reducing components, to enable polymerisation to be initiated and/or accelerated upon contact of the adhesive with the composite restorative.

### FURTHER DESCRIPTION OF EMBODIMENTS

In a preferred embodiment, the self-cure composite restorative of the present invention comprises an amine-free, or substantially amine free, redox initiator system, with at least one oxidising agent and at least one non-amine based reducing agent. The composite restorative may be in a two-part form in a dental kit according to the second aspect of the present invention, and in a two-part combined/mixed form in a dental composition according to the first aspect of the present invention. Advantageously, the redox initiator system enables the dental composition, or the components thereof, to cure or self-cure without the need of a photoinitiator and phtotopolymerisation. Advantageously also, the absence of amine provides excellent long-term colour stability with a dental restoration formed from a composition, kit or method of the present invention.

In a preferred embodiment, both the adhesive and self-cure composite restorative (in one or both part(s)) of the present invention comprise at least one polymerisable component and/or at least one reducing agent.

The at least one polymerisable component in the adhesive and self-cure composite restorative of the present invention may be selected from a monomer, an oligomer, a prepolymer and combinations thereof. In a preferred embodiment, the at least one polymerisable component is one or more of acrylic and/or methacrylic compounds (referred to herein as (meth)acrylic compound(s)), which include (meth)arylic resins, derivatives of the (meth)acrylic compounds and resins of the derivatives. In a further embodiment, the at least one polymerisable component is one or more of acrylate and/or methacrylate compounds (referred to herein as (meth)acrylate compound(s)), which include (meth)acrylate resins, derivatives of the (meth)acrylate compounds and resins of the derivatives. In a preferred embodiment, the polymerisable component in the adhesive and/or composite restorative of the present invention is one or a combination of various monomers, oligomers or prepolymers of (meth)acrylate. In a preferred embodiment, the at least one polymerisable component in the adhesive and/or composite restorative of the present invention has one or more acryloyloxy and/or methacryloyloxy groups (referred to herein as (meth)acryloyoxy group(s)).

In one embodiment, the (meth)acrylate compound(s) of the present invention is/are selected from the group consisting of methacrylate based resins, dimethacrylate based resins, methyl (meth)acrylate, ethyl (meth)acrylate, isopropyl (meth)acrylate, n-butyl (meth)acrylate, isobutyl (meth)acrylate, n-hexyl (meth)acrylate, capryl (meth)acrylate, palmityl (meth)acrylate, tridecyl (meth)acrylate, stearyl (meth)acrylate, allyl (meth) acrylate, lauryl (meth)acrylate, glycerol tri (meth)acrylate, ethylene glycol di (meth)acrylate, diethylene glycol di (meth)acrylate, triethylene glycol di (meth)acrylate, 2-ethylhexyl (meth)acrylate, 2-hydroxypropyl (meth)acrylate, 2-ethoxyethyl (meth)acrylate, glycidyl (meth)acrylate, 3-propanediol di (meth)acrylate, trimethylolpropane tri (meth)acrylate, 1,2,4-butanetriol tri (meth)acrylate, methoxytetraethylene glycol (meth)acrylate, methoxypolyethylene glycol (meth)acrylate, phenoxy-diethyleneglycol (meth)acrylate, phenoxyhexaethyleneglycol (meth)acrylate, 1,4-cyclohexanediol di (meth)acrylate, pentaerythritol tetra (meth)acrylate, phenyl (meth)acrylate, pentaerythritol mono(meth)acrylate, dipentaerythritol mono(meth)acrylate, benzyl (meth)acrylate, dicyclopentenyl (meth)acrylate, isobornyl (meth)acrylate, sorbitol hexa (meth)acrylate, tetrahydrofurfuryl (meth)acrylate, bis [1-(2-acryloxy)]-p-ethoxyphenyldimethylmethane, bis [1-(3-acryloxy-2-hydroxy)]-P-propoxyphenyldimethylmethane, ethoxylated bisphenol A di (meth)acrylate, and trishydroxyethyl-isocyanurate tri (meth)acrylate, urethane (meth)acrylate, ethylene glycol di(meth)acrylate, diethylene glycol di(meth)acrylate, triethylene glycol di(meth)acrylate, tetraethylene glycol di(meth)acrylate, polyethylene glycol di(meth)acrylate, 1,3-butylene glycol di(meth)acrylate, 1,6-hexane diol di(meth)acrylate, neopentyl glycol di(meth) acrylate, tripropylene glycol di(meth) acrylate, polypropylene glycol di(meth)acrylate, glycerol di(meth)acrylate, bisphenol A di(meth)acrylate, bisphenol A glycidyl di(meth)acrylate, ethylene oxide modified bisphenol A di(meth)acrylate, ethylene oxide modified bisphenol A glycidyl di(meth)acrylate, 2,2-bis(4-methacryloxypropoxyphenyl) propane, 7,7,9-trimethyl-4,13-dioxa-3,14-dioxo-5,12-diazahexadecane-1,16-diol di(meth)acrylate, neopentyl glycol hydroxypivalic acid ester di(meth)acrylate, caprolactone modified hydroxypivalic acid neopentyl glycol ester di(meth)acrylate, trimethylol ethane di(meth)acrylate, trimethylol propane di(meth)acrylate, trimethylol methane tri(meth)acrylate, trimethylol ethane tri(meth)acrylate, trimethylol propane tri(meth)acrylate, pentaerythritol tri(meth)acrylate, dipentaerythritol tri(meth)acrylate, pentaerythritol tetra(meth)acrylate, dipentaerythritol tetra(meth)acrylate, dipentaerythritol hexa(meth)acrylate, the reaction product of 3-chloro-2-hydroxypropyl (meth)acrylate and methylcyclohexane diisocyanate, the reaction product of 2-hydroxypropyl (meth)acrylate and methylcyclohexane diisocyanate, the reaction product of 2-hydroxypropyl (meth)acrylate and methylene bis (4-cyclohexylisocyanate), the reaction product of 2-hydroxypropyl(meth)acrylate and trimethylhexamethylene diisocyanate, the reaction product of 2-hydroxyethyl (meth)acrylate and isophorone diisocyanate, and the reaction product of 3-chloro-2-hydroxypropyl (meth)acrylate and isophorone diisocyanate, Bis-Glycidyl methacrylate (Bis-GMA) based resins, 2-hydroxyethyl methacrylate (HEMA) based resins, 1,3-butyleneglycoldimethacrylate (BGD), 2,2-Bis[4-methacroyloxyphenyl]propane (Bis-MA), Bis-GMA, 2,2-Bis[4-(2-methacroyloxy-ethoxy)phenyl]propane (Bis-EMA), 2,2-Bis[4-(3-methacroyloxy-propoxy)phenyl]propane (Bis-PMA), the dimethacrylate derivative of 1,2-cyclohexanedicarboxylic acid (c-HaDMA), the dimethacrylatederivative of 4-cyclohexene-1,2-dicarboxylic acid (c-HeDMA), dimethacrylate monomers containing urethane groups such as UEDMA and TUDMA, ethylene glycol dimethacrylate (EGDMA), trimethylpropanetrimethacrylate (TMPTMA), glycerol dimethacrylate (GDMA), polyethyleneglycoldimethyacrylate (poly-EGDMA), urethane dimethacrylates (UEDMA or UDMA) and triethyleneglycoldimethacrylate (TEGDMA) based resins, di(meth)acrylates of alkylene glycol having 2 to 20 carbon atoms, di(meth)acrylates of the oligomers of alkylene glycol, polyalkyleneglycol di(meth)acrylate, di(meth)acrylate derived from bisphenol A, and combinations thereof.

The (meth)acrylate compounds in the adhesive and/or composite restorative of the present invention may or may not have an acid functional group. In a preferred embodiment, particularly for the at least one polymerisable component in an adhesive of the present invention, the one or more of the (meth)acrylate compounds of the present invention has/have at least one acid functional groups. Advantageously, having at least one acid functional group improves the adhesiveness of the adhesive portion or layer of the present invention and the resultant dental restoration formed. Preferably, the (meth)acrylate compound(s) has/have a plurality of acid functional groups. The acid functional groups may, for example, be carboxylic and/or phosphoric acid groups, or residues thereof (e.g. —COO, —PO(OH)).

In a preferred embodiment, the at least one polymerisable component in the adhesive and/or composite restorative of the present invention having at least one acid group includes, or is selected from: 10-methacryloyloxydecyl dihydrogen phosphate (10-MDP), 4-methacryloxyethyl trimellitic acid (4-MET) and combinations thereof. These (meth)acrylates are particularly preferable in terms of adhesion.

In a preferred embodiment, the content of the (meth)acrylate compound(s) having at least one acid functional group in the adhesive of the dental composition, kit or method of the present invention is in the range of 1 to 70% by weight of the adhesive, more preferably 1 to 60%, 2 to 50% or 3 to 40%, and most preferably 5 to 30% by weight. Advantageously, when the content of the (meth)acrylate having at least one acid group in the adhesive of the present invention is at least between 1% and 60% by weight, the adhesiveness and storage stability of the dental composition and dental kit of the present invention are improved.

In one embodiment, the (meth)acrylates having no acid functional group but can also be used in the adhesive and/or composite restorative of the present invention may, for example, be selected from UDMA, Bis-GMA, TEGDMA, GDMA, HEMA, combinations thereof, and other monomers known in the art without an acid functional group.

The content of the (meth)acrylate compound(s) having no acid group in the adhesive of the dental composition, kit or method of the present invention is preferably in the range of 0.5 to 65% by weight of the adhesive, more preferably 1 to 50% or 2 to 40%, and most preferably 10 to 35% by weight. Advantageously, when the content of the (meth)acrylate(s) having no acid group in the adhesive of the present invention is between 0.5% and 65% by weight, the adhesiveness and storage stability of the dental composition and kit of the present invention are improved.

In a preferred embodiment, the total (or combined) content of the (meth)acrylate compounds (with or without acid functional groups) in a two-part composite restorative of the present invention is preferably in the range of 10 to 80% by weight of the composite restorative (in a combined/mixed form), more preferably 15 to 65%, and most preferably 20 to 55% by weight. Advantageously, when the total content of the (meth)acrylate compounds in the self-cure composite restorative is between 20% and 55% by weight, good viscosity, higher mechanical strength and lower shrinkage of the dental composition or dental restoration formed in situ are achieved.

In a preferred embodiment of the dental composition, kit or method of the present invention, both the adhesive and one (e.g. a second) part of the composite restorative include at least one reducing agent.

In one embodiment, the one or more reducing agents of the present invention are selected from the group consisting of organic reducing agents, inorganic reducing agents and combinations thereof. In a preferred embodiment, the adhesive and one part of the composite restorative each comprises at least two, or two types of, reducing agents, being organic and inorganic reducing agents. In a preferred embodiment, the organic reducing agent may be selected from the group consisting of urea reducing agents (being urea or urea derivatives based reducing agents) and thiourea reducing agents (being thiourea or thiourea derivatives based reducing agents) and combinations thereof.

In a preferred embodiment, both the adhesive and one part of the composite restorative of the present invention have one or more of the same organic reducing agents. In a further embodiment, both the adhesive and composite restorative of the present invention have one or more reducing agents selected from the group consisting of thiourea reducing agents. In another embodiment, only the adhesive or one part of the composite restorative of the present invention comprises one or more thiourea reducing agents. In another embodiment, the adhesive or one part of the composite restorative may comprise one or more further reducing agent(s) in addition to thiourea reducing agent(s), such as other organic reducing agent(s) and/or one or more inorganic reducing agent(s)).

The thiourea reducing agents that may be used in the present invention are not particularly limited. In one exemplary embodiment, both the adhesive and composite restorative of the present invention may comprise one or more reducing agents selected from the group consisting of: thiourea, ethylenethiourea, N-methylthiourea, N-ethylthiourea, N-propylthiourea, N-butylthiourea,N-octyl thiourea, N-laurylthiourea, N-phenylthiourea, N-cyclohexyl Thiourea, N, N-dimethylthiourea, N, N-diethylthiourea, N, N-dipropylthiourea, N, N-di-butylthiourea, N, N-dilaurylthiourea, N, N-diphenylthiourea, N, N-dicyclohexylthiourea, trimethylthiourea, tetramethylthiourea, N-acetylthiourea, allyl thiourea, phenyl thiourea, benzyl thiourea, acetyl thiourea, benzoyl thiourea, octanoyl thiourea, 1-Benzoyl-2-thiourea, 1-allyl-3-(2-hydroxyethyl)-2-thiourea, 1-(2-tetrahydrofufuryl)-2-thiourea, N-tert-butyl-N′-isopropylthiourea, 1-(2-Pyridyl)-2-thiourea, 1,1,3-triphenyl thiourea, 1,1,3-trimethyl thiourea, 2,4-xylyl thiourea, p-tolylsulfonyl thiourea, 1-octyl-3-phenyl thiourea, o-methoxyphenyl thiourea, m-hydroxyphenyl thiourea, 1,1-diallyl thiourea, 1,3-diallyl thiourea, 2-methallyl thiourea, o-methoxybenzyl thiourea, 1-(hydroxymethyl)-3-methyl thiourea, 1,1-dibutyl thiourea, 1,3-dibutyl thiourea, 1-(p-chlorophenyl)-3-methyl thiourea, 1-butyl-3-butyryl thiourea, 1-acetyl-3-phenyl thiourea, 1-methyl-3-(p-vinylphenyl) thiourea, 1-methyl-3-o-tolyl thiourea, 1-methyl-3-pentyl thiourea, 3-methyl-1,1-diphenyl thiourea, 1-acetyl-3-(2-mercaptoethyl) thiourea, (meth)acryloxyalkyl thiourea, 1-allyl-3-methyl thiourea, and combinations thereof.

In a preferred embodiment, the adhesive and one part (e.g. second part) of the composite restorative of the present invention both comprise one or more thiourea reducing agents selected from the group consisting of 1-Benzoyl-2-thiourea, 1-(2-Pyridyl)-2-thiourea and combinations thereof. Advantageously, these thiourea reducing agents have good storage stability.

In a preferred embodiment, the content of the one or more thiourea reducing agent(s) in the adhesive of the present invention is preferably in the range of 0.01 to 5%, more preferably 0.03 to 3%, and most preferably 0.1 to 1% by weight of the adhesive. Advantageously, when the content of the thiourea compound(s) in the adhesive is between 0.03% and 3% by weight, the dental composition and kit of the present invention have good adhesiveness and storage stability.

In a preferred embodiment, the content of the one or more thiourea reducing agent(s) in one part (e.g. a second part) of the composite restorative of the present invention is preferably in the range of 0.01 to 5%, more preferably 0.02 to 2% or 0.1 to 1% by weight of the part of the composite restorative comprising the thiourea reducing agent(s). Advantageously, when the content of the thiourea compound(s) in the relevant part of the composite restorative is between 0.02% and 2% by weight, the dental composition and kit of the present invention show fast setting and good storage stability advantages.

In a preferred embodiment, the adhesive and/or the composite restorative (e.g. in one part) of the dental composition, kit and method of the present invention also comprises at least one polymerisation accelerator. In a preferred embodiment, the at least one polymerisation accelerator is in the form of at least one reducing agent, e.g. at least one inorganic reducing agent. Preferably, the polymerisation accelerator is in the form of a metallic reducing agent. In a further embodiment, the metallic reducing agent is selected from the group consisting of a silver reducing agent, a copper reducing agent, a rhodium reducing agent, a manganese reducing agent, a cobalt reducing agent, a vanadium reducing agent and combinations thereof. In a preferred embodiment, the at least one polymerisation accelerator of the present invention is one or more vanadium compounds (or vanadium reducing agent(s)).

The vanadium compounds that may be used in the present invention are not particularly limited. In one exemplary embodiment, the adhesive and/or one (e.g. a second) part of the composite restorative of the present invention may comprise one or more vanadium reducing agent(s) selected from the group consisting of vanadium pentoxide, vanadyl acetylacetonate, vanadium acetylacetonate, vanadium benzoylacetonate, vanadyl stearate, vanadium naphthenate and combinations thereof. In one particular embodiment, both the adhesive and the composite restorative of the present invention comprise a vanadium compound in the form of vanadyl acetylacetonate. Advantageously, vanadyl acetylacetonate is able to provide a high rate of conversion of the monomers, oligomers and/or prepolymers to polymers, resulting in good adhesiveness of the present invention. In addition, vanadyl acetylacetonate has good storage stability.

In the embodiment that the adhesive also comprises one or more vanadium polymerisation accelerator compound(s), the content of the one or more vanadium compound(s) in the adhesive of the present invention is preferably in the range of 0.001 to 5% by weight of the adhesive, more preferably 0.001 to 2% by weight, and most preferably 0.1 to 1% by weight of the adhesive. When the content of the vanadium compound(s) in the adhesive of the present invention is between 0.001 to 2% by weight, the dental composition and kit of the present invention have good adhesiveness and storage stability.

In the embodiment that one (e.g. a second) part of the composite restorative also comprises one or more vanadium polymerisation accelerator compound(s), the content of the one or more vanadium compound(s) in the relevant part of the composite restorative is preferably in the range of 0.003 to 0.8% by weight of the part of the composite restorative containing the vanadium compound(s), more preferably 0.004 to 0.6% and most preferably 0.005 to 0.5% by weight. Advantageously, when the content of the vanadium compound(s) is in the range of 0.005 to 0.5% by weight, the vanadium compound(s) not only provide(s) a desired level of effect as a polymerisation accelerator but also prevent(s) the part of the composite restorative containing the vanadium compound(s) from self-curing during storage.

In a preferred embodiment of the present invention, one (e.g. a first) part of the composite restorative comprises at least one oxidising agent of the redox initiator system. In a preferred embodiment, the at least one oxidising agent is selected from the group consisting of peroxide oxidising agents, particularly organic peroxides. Exemplary organic peroxides include benzoyl peroxide, lauryl peroxide, ketone peroxide, ketal peroxide, hydroperoxides, diacyl peroxide, dialkyl peroxide, peroxyester and peroxy decarbonate.

In one preferred embodiment, the one or more oxidising agent(s) of the present invention is/are selected from the group consisting of hydroperoxides. Commonly used hydroperoxide oxidising agents in the art may also be used in the present invention. Exemplary hydroperoxide oxising agents that may be used in the present invention include cumyl hydroperoxide, cumene hydroperoxide, t-butyl hydroperoxide, amyl hydroperoxide, p-menthane hydroperoxide, 2,5-dimethylhexane-2,5-dihydro-peroxidase, diisopropylbenzene hydroperoxide, 1,1,3,3-tetramethylbutyl hydroperoxide, and combinations thereof. In a further preferred embodiment, the one or more oxidising agent(s) of the present invention is/are selected from the group consisting of 1,1,3,3-tetramethylbutyl hydroperoxide, cumene hydroperoxide and combinations thereof. Advantageously, these hydroperoxides show better storage stability.

The content of the one or more hydroperoxide oxidising agent(s) in the relevant part of the composite restorative of the present invention is preferably in the range of 0.02 to 5% by weight of the composite restorative part, more preferably in the range of 0.03 to 3% by weight. Advantageously, when the content of the hydroperoxide oxidising agent(s) is in the range of 0.03 to 3% by weight, it not only provides a desired level of redox polymerisation initiator effect but also provides the part of the composite restorative containing the oxidising agent(s) with good storage stability.

In a preferred embodiment of the present invention, the adhesive and composite restorative may either or both include one or more polymerisation inhibitor(s) or stabiliser(s), e.g. to prevent autopolymerisation and improve storage stability of the dental composition, kits or relevant part(s) of the present invention.

The polymerisation inhibitor(s) that may be used in the present invention are not particularly limited. Commonly used polymerisation inhibitors in the art may also be used in the present invention. Exemplary polymerisation inhibitor(s) such as monomethyl ether, hydroquinone, butylated hydroxy toluene (BHT), 2,6-t-butyl-2,4-xylenol, 2,6-di-tert-butyl-p-cresol, 3,5-di-tert-butyl-4-hydroxyanisole, 2,6-di-tert-butyl-4-methoxyphenol, 2,6-di-tert-butyl-4-(dimethylamino)methylphenol; 2,5-di-tert-butyl hydroquinone and the like, or combinations thereof, are suitable. In one embodiment, butylated hydroxy toluene (BHT) is a particularly preferred polymerisation inhibitor (and stabiliser) due to, for example, its safety aspect.

The content of the one or more polymerisation inhibitors in the adhesive, and the composite restorative (or each part of the composite restorative), of the dental composition, kit or method of the present invention is in the range of 0.005 to 5% by weight of the adhesive, by the combined weight of the composite restorative, or by weight of each, or relevant, part of the composite restorative. More preferably, the content of the one or more polymerisation inhibitors in each instance is in the range of 0.01 to 3%, or 0.01 to 2%, by weight. In the instance of the adhesive, advantageously, if the content of the polymerisation inhibitor(s) is 0.01% by weight or more, the storage stability of the dental composition is improved; and if the content of the polymerisation inhibitor(s) is 3% by weight or less, the adhesiveness is improved.

The adhesive in the dental composition, kit and method of the present invention may further comprise one or more ingredients selected from the group consisting of water; at least one water-soluble organic solvent; at least one filler material; other desired elements such as thickening agent(s) or coupling agent(s); and combinations thereof.

The self-cure composite restorative (one or both parts) in the dental composition, kit and method of the present invention may further comprise one or more ingredients including water; at least one filler material; at least one pigment (or shade pigment); other desired elements such as thickening agent(s) or coupling agent(s); and combinations thereof.

The thickening and coupling agents in the present invention are not particularly limited. For example, fumed silica thickening agents and silane coupling agents used in the art can also be used in the present invention.

Advantageously, it has been found that the surface wetting and decalcification of a tooth to be restored using a dental composition, kit or method of the present invention are improved if the adhesive comprises water. This, in turn, leads to improved adhesiveness (including of the dental composition or restoration formed in situ of an oral or tooth cavity).

The content of water in an adhesive according to the dental composition, kit or method of the present invention is preferably in a range of 1 to 40% by weight of the adhesive, more preferably 5 to 30% by weight. Advantageously, when the content of water in the adhesive is 1% or more by weight, the solubility of a smear layer on the tooth surface, the decalcification property of the dentin and the permeability of the dentin are improved; and when the content of water in the adhesive is 40% or less by weight, the uniformity of the dental composition or dental restoration formed in situ is improved.

In a further embodiment, the adhesive in the dental composition, kit or method of the present invention may comprise at least one water-soluble organic solvent in addition to including water. The one or more water-soluble organic solvent that may be used in the present invention are not particularly limited, provided it has volatility. Exemplary organic solvents include acetone, methanol, ethanol, 1-propanol, 2-propanol, 1-butanol, 2-methyl-2-propanol, isopropyl alcohol, t-butyl alcohol, tetryhydrofuran and/or methyl ethyl ketone (MEK). In a preferred embodiment, the at least one water-soluble organic solvent in an adhesive according to the present invention is selected from the group consisting of ethanol, 2-propanol, e-methyl-2-propanol, acetone, tetrahydrofuan, methyl ethyl ketone and combinations thereof. In a further embodiment, the water-soluble organic solvent is acetone and/or methyl ethyl ketone.

The content of the one or more water-soluble organic solvent(s) in the embodiment of the adhesive containing the solvent(s) is preferably in the range of 5 to 60% by weight of the adhesive, and more preferably 10 to 40% by weight. Advantageously, when the content of the water-soluble solvent in the adhesive is 5% or more by weight, the uniformity of the dental composition or dental restoration formed in situ is improved; and when the content of the water-soluble organic solvent is 60% or less by weight, the adhesiveness is improved.

In a preferred embodiment of the dental composition, kit or method of the present invention, the adhesive and/or composite restorative (one or both parts) may further comprise at least one filler material. The at least one filler material that may be used in the present invention is not specifically limited. Common filler materials used in the art may also be used in the present invention. In a preferred embodiment, the one or more filler material(s) according to the present invention may be selected from inorganic fillers, organic fillers, polymer- or fibre-based fillers and combinations thereof. The inorganic fillers and organic fillers may be in the form of inorganic and organic particles. The inorganic filler materials may be treated with a surface treatment agent such as a silane coupling agent, if required.

In one embodiment, the at least one filler material in the adhesive and/or composite restorative of the present invention may be selected from the group consisting of crystalline silica, amorphous silica, fumed silica, ytterbium trifluoride, ceramic powders, graphite, polymeric filler materials (such as cellulose, polyesters, polyolefins, polyacrylates, polyurethanes, vinyl esters, styrenes, polysulphones, polysulphides, polyacetals and polycarbonates), glass powders (which may include barium glass, strontium glass, fluoroaluminosilicate glass), silicate glass, quartz, zinc oxide, barium sulfate, barium silicate, strontium silicate, barium borosilicate, strontium borosilicate, borosilicate, lithium silicate, deammoniated calcium phosphate, alumina, zirconia, tin oxide, titania, apatites, calcium silicate based fillers, hydroxyapatites, barium sulfate, bismuth subcarbonate, and combinations thereof.

The content of the filler material(s) in the composite restorative is preferably in the range of 10 to 80% by weight of the composite restorative (two-parts combined/mixed), and more preferably 20 to 55% by weight. Advantageously, when the content of the filler material(s) in the composite restorative of the present invention is between 20% and 55% by weight, good viscosity, higher mechanical strength and lower shrinkage of the dental composition or dental restoration formed are achieved.

In a preferred embodiment, the adhesive in the first, second or third aspect of the present invention is a one-part adhesive, which may be directly applied to a prepared tooth cavity requiring restoration (without the need of an external pre-mixing step).

In a preferred embodiment, the one-part adhesive of the present invention comprises at least one polymerisable component (e.g. at least one (meth)acrylate compound) and at least one reducing agent (e.g. at least one thiourea reducing agent). In a further preferred embodiment, the one-part adhesive of the present invention further comprises at least one polymerisation accelerator (e.g. at least one vanadium compound). In a further embodiment, the one-part adhesive of the present invention further comprises one or more ingredients selected from the group consisting of: at least one polymerisation inhibitor (e.g. at least BHT), at least one filler material (e.g. inorganic fillers), water, at least one water-soluble organic solvent (e.g. acetone and MEK), and combinations thereof.

In a preferred embodiment, the self-cure composite restorative defined in the first, second or third aspect of the present invention has a first part comprising at least one oxidising agent (e.g. at least one peroxide or hydroperoxide oxidising agent) and at least one polymerisable component (e.g. at least one (meth)arylate compound selected from (meth)acrylate monomers, oligomers and prepolymers). In a further embodiment, the first part of the self-cure composite restorative further comprises one or more ingredients selected from the group consisting of: at least one polymerisation inhibitor (e.g. at least BHT), at least one filler material (e.g. at least one inorganic filler), at least one pigment, at least one thickening agent, at least one coupling agent, and combinations thereof.

In a preferred embodiment, the self-cure composite restorative defined in the first, second or third aspect of the present invention has a second part comprising at least one reducing agent (e.g. at least one organic reducing agent, such as at least one thiourea reducing agent) and at least one polymerisable component (e.g. at least one (meth)arylate compound selected from (meth)acrylate monomers, oligomers and prepolymers). In a preferred embodiment, the second part of the self-cure composite restorative further comprises at least one polymerisation accelerator (e.g. at least one vanadium compound or reducing agent). In a further embodiment, the second part of the self-cure composite restorative further comprises one or more ingredients selected from the group consisting of: at least one polymerisation inhibitor (e.g. at least BHT), at least one filler material (e.g. at least one inorganic filler), at least one pigment, at least one thickening agent, at least one coupling agent, and combinations thereof.

In a preferred embodiment, the first and second part of the composite restorative each comprises at least one thickening agent to provide or maintain a paste form. In one embodiment, components (essential and/or optional) of the first part and second part of the composite restorative can be blended into the respective resin/paste.

In a preferred embodiment, the ratio of the mass/weight of the first part of the compsite restorative resin/paste to second part of the restorative resin/paste is preferably in the range of 5:1 to 1:5, more preferably 3:1 to 1:3, 2:1 to 1:2, or 1:1, to ensure good or sufficient redox polymerisation.

In one embodiment, a thickening agent may also be included in the adhesive of the dental composition, kit and method of the present invention to maintain a desired viscosity.

In a preferred embodiment, the self-cure composite restorative, as a whole, comprises one or more (meth)acrylate compound(s) and an amine-free, or substantially amine free, redox polymerisation initiation or initiator system. The redox initiator system comprises at least one non-amine based oxiding agent (e.g. at least one peroxide or hydroperoxide compound) and at least one non-amine based reducing agent (e.g. at least one thiourea compound). The self-cure composite restorative preferably further comprises at least one polymerisation accelerator (e.g. a vanadium compound). The self-cure composite restorative may further comprise at least one filler material and/or at least one polymerisation inhibitor. The self-cure composite restorative may further comprise one or more ingredients selected from the group consisting of: at least one water-soluble organic solvent, at least one pigment, at least one thickening agent, at least one coupling agent, and combinations thereof.

The self-cure restorative may be in a ‘two-part combined’ form in the dental composition according to the first aspect of the present invention, and a two-part (or two-part separately packaged) form in the dental kit according to the second aspect of the present invention. The composite restorative of the present invention may be in liquid, powder, resin/paste, or a combination of these forms. In one embodiment, the dental kit according to the second aspect of the present invention may comprise a composite restorative that are separately packaged, or packaged in a compartmentalised container such as a compartmentalised capsule, tray or syringe, with one part of the composite restorative in a same, or in a different form, to the other part.

In one embodiment of a dental kit according to the second aspect of the present invention, the kit may comprise three separately packaged parts, including an adhesive and the self-cure composite restorative in two separate parts.

In use, there is provided a method of applying a dental composition to a tooth cavity according to the third aspect of the present invention, in which a polymerisable adhesive of the present invention may be applied to a tooth cavity. In one embodiment, the method may optionally comprise an earlier step of preparing the tooth cavity, e.g. removing decayed component(s) and/or cleaning a cavity interface (e.g. by blowing air/water over the interface). The adhesive may then be applied to the prepared cavity, or cavity interface, to form an adhesive layer. In an embodiment in which the adhesive comprises at least one organic solvent, prior to applying a self-cure composite restorative according to the present invention, the adhesive layer may be blown with clean, oil-free air to remove any organic solvent. Once the adhesive layer is applied (and optionally with any organic solvent removed where relevant), a self-cure composite restorative of the present invention may then be applied to the adhesive layer. In a preferred embodiment, a first part of the self-cure composite restorative (e.g. comprising the least one oxidising agent of the redox initiator system) is mixed with a second part of the composite restorative (e.g. comprising the at least one reducing agent), the mixed/combined composite restorative may then be applied over the adhesive layer. In a preferred embodiment where the adhesive of the present invention already comprises at least one reducing agent, the contact of the composite restorative with the adhesive layer cures the adhesive rapidly, ensuring a stable bond is established quickly at the restoration/cavity interface. In comparison, the slower curing rate in the bulk of the self-cure composite allows volumetric shrinkage to occur mainly at a free surface, hence reducing the residual stress caused by polymerisation shrinkage at the restoration/cavity interface. In one embodiment of the method, a mold may be arranged on the adhesive layer and filled with a two-part combined/mixed composite restorative. In any instance, whilst the composite restorative component of the present invention may self-cure (e.g. depending on the levels of polymerisation accelerator and/or inhibitor in the composite restorative), it cures at a slower rate when not in contact with the adhesive.

In a preferred embodiment, the adhesive and self-cure composite restorative of a method according to the third aspect of the present invention are the same as those in the first or second aspect of the present invention, or have components/ingredients as defined in any one of the embodiments of the adhesive and self-cure composite restorative of the present invention. In one embodiment, the method of applying a dental composition of the first aspect of the present invention to a tooth cavity involves the use of a dental kit according to the second aspect of the present invention.

In one embodiment in which the self-cure composite restorative is in a two-part paste/paste form, a method according to the third aspect of the present invention may involve applying a mixed/combined self-cure composite restorative resin/paste to the adhesive layer. In one embodiment, the two parts of the self-cure composite restorative may be conveniently mixed and delivered in one step using existing dental mixing devices comprising a dual barrel (with one barrel containing one part of the restorative) and an auto-mixing tip.

In another embodiment where the composite restorative is in a two-part powder/liquid form, a compartmentalised capsule (with a delivery tip) may be used to contain, mix and deliver the two parts in a mixed form. In another embodiment, the two parts may be separately packaged, with a portion of each part is placed in a tray and simply mixed with a spatula.

Advantageously, the adhesive of the present invention differs from commercially available bonding agents in at least that the adhesive of the present invention does not contain any photoinitiator/photoinitiation system, but rather it is (rapidly) cured, in situ, upon direct contact with the subsequent application of the self-cure composite restorative over the adhesive or adhesive layer. Therefore, the present method of applying a dental composition to a tooth cavity may only be a simple two-step process of applying a polymerisable adhesive first to a tooth cavity interface, then applying a self-cure composite restorative of the present invention (in an already mixed form) over the adhesive layer.

In one embodiment of a method according to a fourth aspect of the present invention, there is provided a method of restoring a tooth using a dental composition or a dental kit according to the first or second aspect of the present invention, with the adhesive and self-cure composite restorative having components/ingredients as defined in any one of the embodiments of the adhesive and composite restorative described herein. For example, the adhesive may comprise at least one (meth)acrylate compound and at least one reducing agent (e.g. at least one thiourea reducing), and the self-cure composite restorative may comprise at least one (meth) acrylate compound, at least one oxidising agent (e.g. hydroperoxide) and at least one reducing agent (e.g. at least one thiourea reducing agent). The adhesive and/or the self-cure restorative may further comprise at least one polymerisation accelerator (e.g. at least one vanadium compound) and/or one or more ingredients selected from the group consisting of: at least one polymerisation inhibitor (e.g. at least BHT), at least one filler material (e.g. inorganic fillers), water, at least one water-soluble organic solvent (e.g. acetone and MEK), and combinations thereof. A dental composition, or a dental kit, of the present invention may serve to provide one or more dental restoration(s) in a person having one or more tooth cavities.

In one embodiment of a method according to a fifth aspect of the present invention, there is provided a method of replacing, e.g. a conventional dental amalgam material, for tooth restoration by the use of a dental composition according to the first aspect of the present invention, or a dental kit according to the second aspect of the present invention, which is able to form a dental restoration in situ free of the amalgam material. In one embodiment, the method according to the fifth aspect of the present invention may include providing one, preferably a plurality of, dental kits according to the second aspect of the present invention, to replace one or more commercially provided dental amalgam product(s) in a dental restoration procedure, in dental industry in general, or in a dental consumables market.

In accordance with a sixth aspect of the present invention, there is provided a method of replacing an existing dental amalgam filling within a tooth cavity by: a) removing the dental amalgam filling from the tooth cavity, and b) forming a dental composition according to the first aspect of the present invention, in situ, using a dental kit according to the second aspect of the present invention.

The adhesive and self-cure composite restorative in the methods according to the fifth and sixth aspect of the present invention are the same as those in the first or second aspect of the present invention, or have components/ingredients as defined any one of the embodiments of the adhesive and self-cure composite restorative of the present invention.

In accordance with a seventh aspect of the present invention, there is provided a use of a dental kit according to the second aspect of the present invention in the preparation of a dental composition for restoring a tooth structure, with the dental kit having adhesive and self-cure composite restorative components/ingredients as defined any one of the embodiments of the adhesive and composite restorative of the present invention. In a preferred embodiment, the dental kit is used to form a dental composition in situ. In a preferred embodiment, the dental composition is that or those of the first aspect of the present invention in accordance with the embodiments described herein.

Advantageously, an adhesive and a self-cure composite restorative of the present invention is able to combine, in situ, to create a stable, strong and durable alternative restorative composition or system to dental amalgam (or to existing light-cure restorative compositions, materials or systems in the art).

Advantageously also, the amalgam-alternative dental composition and kit of the present invention provide a restorative system that exhibits strong adhesion to the hard dental tissues, excellent strength/durability and colour stability, whilst being easy to place or use clinically with minimal specialist equipment. Advantageously also, like dental amalgam, the present restorative system (composition or kit) does not require light-curing to harden the filling material.

Further non-limiting description of the adhesive and self-cure composite restorative of the present invention is provided below, by way of examples and comparative examples that follow.

### EXAMPLES

### Example 1

Exemplary adhesives A1 to A3 in accordance with the present invention and adhesive Comparative Example 1.

TABLE 1

| | Example | Example | Example | |
|---|---|---|---|---|
| | A1 | A2 | A3 | |
| | Adhesive | Adhesive | Adhesive | Comparison |
| | for self- | for self- | for self- | 1 |
| | cure | cure | cure | Light-cure |
| | restorative | restorative | restorative | adhesive |
| | (wt %) | | | SDI |
| Acetone | | 30 | 29 | ZipBond |
| MEK | 27.3 | | | Universal |
| Deionised Water | 19 | 19 | 20 | |
| HEMA | 20 | 15 | 20 | |
| UDMA | 10 | 20 | 10 | |
| MDP | 10 | 5 | 10 | |
| 4-META | 10 | 10 | 10 | |
| 1-(2-Pyridyl)-2- | | 0.2 | | |
| thiourea | | | | |
| 1-Benzoyl-2- | 0.2 | | 0.2 | |
| thiourea | | | | |
| VAcAc | 0.2 | 0.5 | 0.5 | |
| BHT | 0.3 | 0.3 | 0.3 | |
| Aerosil R812 | 3 | | | |
| Total | 100 | 100 | 100 | |

### Example 2

Exemplary self-cure composite restoratives RC1 to RC3 according to the present invention and resin composite Comparative Example 1.

TABLE 2

| | Example | Example | Example | Comparison |
|---|---|---|---|---|
| | RC1 | RC2 | RC3 | 1 |
| | Powder/ | Paste/Paste | Paste/Paste | Light-cure |
| | Liquid | in | in | resin |
| | in Capsule | capsule | syringe | composite |
| | First Part (wt %) | | | |
| Barium glass | | 53 | 53 | SDI |
| powder | | | | Aura |
| YbF3 | | 7 | 7 | Easyflow |
| UDMA | 64 | 16.58 | 18.68 | |
| TEGDMA | 29.4 | 16.58 | 18.68 | |
| MDP | 6 | 4 | | |
| Aerosil R202 | | 2 | 2 | |
| CHPO | 0.6 | | 0.6 | |
| TMBH-L | | 0.8 | | |
| BHT | | 0.04 | 0.04 | |
| Total | 100 | 100 | 100 | |
| | Second Part (wt %) | | | |
| Barium glass | | 53 | 53 | |
| powder | | | | |
| FAS glass | 98.85 | | | |
| powder | | | | |
| YbF3 | | 7 | 7 | |
| UDMA | | 18.81 | 16.81 | |
| TEGDMA | | 18.81 | 16.81 | |
| MDP | | | 6 | |
| Aerosil R202 | 1 | 2 | | |
| 1-(2- | | | 0.3 | |
| Pyridyl)-2- | | | | |
| thiourea | | | | |
| 1-Benzoyl-2- | 0.08 | 0.3 | | |
| thiourea | | | | |
| BHT | | 0.04 | 0.04 | |
| VAcAc | 0.06 | 0.03 | 0.03 | |
| Pigment | 0.01 | 0.01 | 0.01 | |
| Total | 100 | 100 | 100 | |
| First Part/ | 1/3 | 1/1 | 1/1 | |
| Second Part | | | | |
| mixing ratio | | | | |

In TABLES 1 and 2 above:
MEK: Methyl ethyl ketone
HEMA: Hydroxyethylmethacrylate
UDMA: Urethane Dimethacrylate
MDP: 10-Methacryloyloxydecyl dihydrogen phosphate
4-META: 4-Methacryloxyethyl trimellitate anhydride. 4-Methacryloxyethyl trimellitic acid (4-MET) is produced by the reaction of 4-META and water incorporated
VacAc: Vanadyl acetylacetonate
BHT: Butylated hydroxytoluene
Aerosil R812: Fumed silica
Aerosil R202: Fumed silica
Barium glass powder: 2.8 μm (D50), 2% Silane treated barium glass
FAS glass powder: 4.0 μm (D50), 2% Silane treated fluoroaluminosilicate glass
YbF₃: Ytterbium(III) fluoride
TEGDMA: Triethylene glycol dimethacrylate
CHPO: Cumene Hydroperoxide
TMBH-L: 1,1,3,3-Tetramethylbutyl hydroperoxide
Pigment: Mixture of iron oxide and titanium oxide (Trade name: RED IRON OXIDE BC, Cosmetic YELLOW OXIDE C33-8073 and TIDIOX8100)

### Example 3

Various tests including Shear Bond Test, Flexural Test and Opacity Test were performed on exemplary dental compositions according to the present invention, formed from adhesives A1-A3 and self-cure composite restoratives RC1-RC3 shown in Examples 1 and 2; and on a comparative light-cure dental composition formed from adhesive Comparative Example 1 and resin composite Comparative Example 1.

### Shear Bond Test

To evaluate adhesiveness of dental compositions/adhesives according to the present invention, shear bond test in accordance with ISO 29022:2013 (Dentistry—Adhesion-Notched-edge shear bond strength test), was conducted on the dental compositions of the present invention and on the comparative light-cure dental composition.

Bovine teeth were polished with wet abrasive paper #600 and the flat polished surface was used as the adherend (test) surface.

To form dental compositions according to the present invention (with adhesives A1-A3 and restoratives RC1-RC3), in each instance, an adhesive was applied to the prepared bovine teeth surface, and then gently air-blown for 5 seconds. Ultradent mold was then mounted on the bonding/prepared surface and filled with the mixed resin/paste composite restorative. Specimens (n=5) were stored at 37° C. oven for 1 hour and immersed in water at 37° C. for 23 hours followed by shear bond test using a universal testing machines INSTRON 5942. The determination criteria of adhesiveness are as follows: Pass—When the average value of shear strength is 10 MPa or more; Fail—When the average value of shear strength is less than 10 MPa.

For the comparative light-cure dental composition, the adhesive was applied to a prepared bovine teeth surface, and then gently air-blown for 5 seconds. The adhesive was light-cured with WSDI radii xpert for 10 seconds. Ultradent mold was mounted on the bonding/prepared surface, and filled with resin composite Comparative Example 1 followed by light-curing for 20 seconds. Specimens (n=5) were stored at 37° C. oven for 1 hour and immersed in water at 37° C. for 23 hours followed by shear bond test using a universal testing machine INSTRON 5942.

### Flexural Test

To evaluate the mechanical strength of the dental composition/self-cure composite restorative of the present invention, flexural test was conducted in accordance with ISO 4049:2019 (Polymer-based restorative materials).

### Opacity Test

Opacity was measured to evaluate the aesthetics of the dental composition/self-cure composite restorative of the present invention. Test specimens were prepared in accordance with ISO 7.13 and ISO 4049:2019 (Polymer-based restorative materials) followed by opacity measurement with a colour measurement device x-rite SP64 after 1 hour in a 37° C. oven.

TABLE 3

| | | | | Comparison |
|---|---|---|---|---|
| Adhesive | Example A1 | Example A2 | Example A3 | 1 |
| Restorative | Example | Example | Example | Comparison |
| material | RC1 | RC2 | RC3 | 1 |
| | | | | Light-cure |
| | Self-cure composite restorative | | | resin system |
| Shear bond | 23.3 (4.6) | 21.2 (3.4) | 19.4 (4.8) | 14.8 (2.0) |
| strength to | | | | |
| enamel | | | | |
| (MPa) | | | | |
| Shear bond | 27.1 (7.8) | 19.7 (1.8) | 23.7 (3.5) | 24.1 (3.8) |
| strength to | | | | |
| dentin | | | | |
| (MPa) | | | | |
| Flexural | 129.3 (8.4) | 117.5 (15.7) | 126.1 (18.3) | 120.6 (17.0) |
| strength | | | | |
| (MPa) | | | | |
| Opacity | 61.5 | 59.6 | 61.5 | 60.3 |
| (%) | | | | |
| Light-cure | No | No | No | Yes |
| required for | | | | |
| adhesive | | | | |
| Light-cure | No | No | No | Yes |
| required for | | | | |
| restorative | | | | |
| material | | | | |
| Incremental | No | No | No | Yes |
| filling | | | | |
| technique | | | | |
| required | | | | |
| Bulk-fill | Yes | Yes | Yes | No |
| technique | | | | |
| applicable | | | | |

( ): Standard deviation

From the Shear Bond Test, Table 3 shows that the self-cure composite restorative of the present invention (in particular adhesives A1 to A3) exceeded the bond strength criteria (>10 MPa). These bond strengths are similar or significantly stronger than the comparative light-cure resin composite which clinically requires the incremental filling technique due to the light-cure shrinkage stress. This filling technique is time consuming. Meanwhile the bulk-fill technique (which is much more efficient) is clinically recommendable for the self-cure composite restorative of the present invention (RC1 to RC3) due to its lower shrinkage formed in situ. The Shear Bond Test also shows that an adhesive according to the presentation functions highly effectively as an adhesive, primer or bonding agent.

From the Flexural Test, Table 3 shows that the mechanical strength, measured as flexural strength, showed no significant difference between the self-cure composite restorative of the present invention (Examples A1 to A3+RC1 to RC3) and the commonly used light-cure resin system (adhesive Comparative Example 1). Importantly, the mechanical strength of the dental compositions in accordance with the present invention is comparable to that of typical dental amalgam.

For the Opacity Test, natural human teeth have moderate translucency, and opacity value represents degree of translucency. The self-cure composite restorative of the present invention (Examples RC1 to RC3) have similar opacity values with the aesthetic light-cure resin system (resin composite Comparative Example 1).

Thus, the present invention has achieved an amalgam-alternative composite restorative solution (including the dental composition of the present invention) that can be applied in a simple two-step process to provide a desirably aesthetic, strong, long-lasting dental restoration with strong bond strength that can be used as a substitute for restorative dental procedures typically requiring amalgam. The present invention is also able to replace existing light-cure restorative materials. The present invention also provides an easy-to-use dental kit and easy methods for forming a dental composition or restoration within a tooth cavity.

Modifications and variations as would be apparent to a skilled addressee are deemed to be within the scope of the present invention.

1. A dental composition comprising:
a polymerisable adhesive free of a photoinitiator, and
a self-cure composite restorative including a redox initiator system;
wherein the dental composition cures upon contact between the self-cure composite restorative and the adhesive, to thereby form a dental restoration.

2. A dental kit with pre-packaged parts for restoring a tooth cavity, the kit comprising:
a polymerisable adhesive free of a photoinitiator, and
a self-cure composite restorative including a redox initiator system.

3. The dental composition according to claim 1 wherein the redox initiator system is a substantially amine-free redox initiator system.

4. The dental composition of claim 3, wherein the self-cure composite restorative is in a two-part or two-part combined form, with one part comprising at least one oxidising agent of the redox initiator system and another part comprising at least one reducing agent of the redox initiator system.

5. The dental composition according to claim 3, wherein the at least one oxidising agent is selected from the group consisting of peroxide oxidising agents.

6. The dental composition according to claim 3, wherein the at least one reducing agent is selected from the group consisting of organic reducing agents, including thiourea reducing agents.

7. The dental composition according to claim 3, wherein both the adhesive and the self-cure composite restorative comprise at least one polymerisable component selected from the group consisting of (meth)acrylate compounds, including (meth)acrylate monomers, oligomers and/or prepolymers.

8. The dental composition according to claim 3, wherein the self-cure composite restorative further comprises at least one polymerisation accelerator.

9. The dental composition according to claim 8, wherein the adhesive also comprises at least one polymerisation accelerator.

10. The dental composition according to claim 8, wherein the at least one polymerisation accelerator is selected from the group consisting of metallic reducing agents, including vanadium reducing agents.

11. The dental composition according to claim 8, wherein the content of the polymerisation accelerator(s) in the self-cure composite restorative is in the range of 0.005 to 0.5% by weight of the adhesive.

12. The dental composition according to claim 9, wherein the content of the polymerisation accelerator(s) in the adhesive is in the range of 0.001 to 2% by weight of the adhesive.

13. The dental composition according to claim 3, wherein the adhesive and/or the self-cure compositive restorative further comprises at least one polymerisation inhibitors.

14. The dental composition according to claim 13, wherein the content of the polymerisation inhibitor(s) in the adhesive is in the range of 0.01 to 3% by weight of the adhesive.

15. The dental composition according to claim 13, wherein the content of the polymerisation inhibitor(s) in the self-cure composite restorative is also in the range of 0.01 to 0.3% by weight of the composite restorative.

16. The dental composition according to claim 3, wherein the adhesive further comprises one or more ingredients selected from the group consisting of: water, at least one water-soluble organic solvent, at least one filler material, at least one thickening agent, at least one coupling agent, and combinations thereof.

17. The dental composition according to claim 3, wherein the self-cure composite restorative further comprises one or more ingredients selected from the group consisting of: water, at least one filler material; at least one pigment, at least one thickening agents, at least one coupling agent, and combinations thereof.

18. The dental composition according to claim 16, wherein the self-cure composite restorative further comprises one or more filler material(s).

19. The dental composition according to claim 3, wherein the dental composition, or a dental restoration, is formed in situ within an oral or tooth cavity.

20. A method of applying a dental composition to a tooth cavity, the method comprising the steps of:
applying a polymerisable adhesive free of a photoinitiator to the cavity to form an adhesive layer; and
applying a self-cure composite restorative including a redox initiator system over the adhesive layer.

21. A method according to claim 20, wherein the self-cure composite restorative comprises at least one oxidising agent and at least one reducing agent for the redox initiator system.

22. A method according to claim 21, wherein the adhesive also comprises at least one reducing agent, to enable the adhesive to cure upon contact with the self-cure composite restorative.

23. A method of restoring a tooth structure using a dental composition according to claim 3.

24. A method of replacing a dental amalgam material for tooth restoration by the use of a dental composition according to claim 3, which is able to form or provide a dental restoration in situ free of the amalgam material.

25. A method of replacing a dental amalgam filing within a tooth cavity by: a) removing the dental amalgam filling from the tooth cavity, and b) forming a dental composition according to claim 3 within the same tooth cavity.

26. The dental kit according to claim 2, wherein, in use, the polymerizable adhesive and self-cure composite restorative together comprise a dental composition for restoring a tooth structure, and the dental composition cures upon contact between the self-cure composite restorative and the adhesive, to thereby form a dental restoration on the tooth structure.
